(19) United States
(12) Patent Application Publication
(10) Pub. No.: US 2010/0222750 A1
(43) Pub. Date: Sep. 2, 2010

(54) REPLENISHABLE DRUG DELIVERY IMPLANT FOR BONE AND CARTILAGE

(75) Inventor: Joseph S. Cheng, Nashville, TN (US)

Correspondence Address: Connolly Bove Lodge & Hutz LLP, Suite 1100, 1875 Eye Street, NW, Washington, DC, 20006, US

(73) Assignee: Vanderbilt University, Nashville, TN (US)

(21) Appl. No.: 12/715,058

(22) Filed: Mar. 1, 2010

Related U.S. Application Data
(60) Provisional application No. 61/156,294, filed on Feb. 27, 2009.

(51) Int. Cl. A61M 31/00; A61B 17/70

(52) U.S. Cl. 604/288.04; 606/246

(57) ABSTRACT

A spinal implant comprising one or more integrated walls spaced apart to define an interior cavity configured to retain bone graft material, and having at least one aperture providing a pathway between the interior cavity and an exterior environment of the implant; wherein at least one of the one or more walls has a lumen therein, the lumen terminating at a post-operatively accessible refill port and at one or more drug delivery ports disposed in a surface of the at least one wall, and configured to allow drugs to flow from the refill port to the one or more delivery ports.

### CROSS-REFERENCE TO RELATED APPLICATIONS

The present application claims the benefit of U.S. Provisional Patent Application 61/156,294, filed Feb. 27, 2009, which is hereby incorporated by reference herein.

### BACKGROUND

1. Field of the Invention

The present invention relates generally to medical implants, and more specifically, to a replenishable drug delivery implant for bone and cartilage.

2. Related Art

Certain conditions, defects, deformities and injuries may lead to structural instabilities, in a patient's bone, cartilage or other connective tissue. Such structural instability is particularly problematic in a patient's spinal column due to the potential for nerve or spinal cord damage, pain and other manifestations. FIG. 1 is a perspective view of a segment 100 of a human spinal column. An individual's spinal column (sometimes referred to as the vertebral column) extends from the person's skull (not shown) to the pelvis (also not shown) and consists of 33 individual bones known as vertebrae 102. Two such vertebrae 102 are illustrated in FIG. 1. Between each vertebra 102 is a soft, gel-like cushion known as an intervertebral disc 104 which absorbs pressure and prevents vertebrae 102 from contacting each other. There are two such intervertebral discs 104 illustrated in FIG. 1. Each vertebra 102 is held to other vertebrae in the spinal column by ligaments (not shown) which also connect t vertebrae 102 to the individual's muscles. Additional tendons (not shown) also fasten muscles to vertebrae 102.

Each vertebra 102 comprises a centrum or vertebral body 106 comprised of dense cortical bone forming the anterior portion of vertebra 102. Vertebral bodies 106 collectively provide structural support to the spinal column. Posterially extending from vertebral body 106 is a spinous process 122 and two transverse processes 120 on opposing lateral sides of spinous process 122. The portion of vertebra 102 which extends between transverse processes 120 and which is disposed between transverse processes 120 and vertebral body 106 is referred to as pedicle 118. Processes 120,122 add structural rigidity, assist in articulation of vertebrae 102 in conjunction with the individual's ribs (not shown), and serve as muscle attachment points.

Each vertebra 102 further comprises lamina 110 which form the walls of spinal canal 112. Extending through spinal canal 112 is spinal cord 114.

Damage and structural instability to a patient's spine may occur in a variety of circumstances. One notable cause of structural instability in an individual's spinal column is due to bone metastases associated with advancement of cancer cells originating at other locations in the individual's body. Spinal metastasis occurs in 5-10% of all patients who suffer from cancer. Barron, K. D. et al., Neurology 9:91-106 (1959). Furthermore, autopsy studies have found metastatic involvement of the spinal column in 90% of patients with prostate cancer, in 75% of patients with breast cancer, 45% of patients with lung carcinoma, 55% of patients with melanoma, and 30% of patients with renal carcinoma. Lenz, M. et al., Ann Surg 93:278-293 (1931); Sundaresan N, et al., Tumors of the Spine: Diagnosis and Clinical Management. Philadelphia: WB Saunders: pp 279-304 (1990); Wong, D. A. et al., Spine, 15:1-4 (1990).

About 10% of patients who suffer from spinal metastasis will subsequently develop spinal cord compression. Schaberg J. et al., Spine 10:19-20 (1985); Sundaresan N, et al., Neurosurgery, 29:645-650 (1991). The metastatic spinal lesions affect vertebral body 102 and pedicle 118 in approximately 85% of the patients suffering from spinal metastasis. Riaz et al., supra. The distribution of the metastatic lesions according to the level of vertebrae in various spinal segments is: thoracic spine 70%, lumbar spine 20% and cervical spine 10%. Barron et al., supra; Gilbert R W, et al., Ann Neurol, 3:40-51 (1978). Typically, the posterior region of vertebral body 102 is invaded first, with the anterior region, lamina, and pedicles invaded at a later time. Adams M, et al., Contemp Neurosurg, 23:1-5 (2001).

The treatment of spinal metastasis is primarily palliative except in rare circumstances. Available treatments include chemotherapy, radiotherapy, hormonal therapy and/or surgery. Surgery is typically used in patients suffering from spinal metastases that include occurrences of a radio-resistant tumor, spinal instability, progressive deformity or neurologic compromise, significant neurologic compression due to retropulsed bone or bone debris, and intractable pain unresponsive to nonoperative therapies. Tomita K, et al., Spine, 26(3):298-306 (2001).

Surgical treatment for spinal metastases may involve discectomy (i.e., surgical removal of an intervertebral disc 104), corpectomy (i.e., surgical removal of a portion of vertebral body 106), and vertebrectomy (i.e., surgical removal of an entire vertebra 102). Thongtrangan I, et al., Neurosurg Focus, 15(5) (2003). Regardless of whether discectomy, corpectomy or vertebrectomy is performed, reconstruction is required to stabilize the spinal column. Reconstruction traditional uses bone grafts and/or bone cement, alone or in combination with various implants.

Certain procedures use implants positioned in the patient's spinal bone or cartilage, collectively and generally referred to as spinal implants herein, to effect or augment the biomechanics of a patient's spine. One common type of spinal implant that is used following corpectomy or vertebrectomy is a vertebral body implant which is positioned in a patient's vertebral body 106. Currently, there are a wide number of available vertebral implants of varying design and material. One class of vertebral body implant is configured to directly replace the excised vertebra/ae. Another class of vertebral body implant is configured for insertion into the intervertebral space in a collapsed state and then expanded to contact adjacent vertebrae. The use of expandable implants may be advantageous since a smaller incision is required to insert the implant into the intervertebral space. Additionally, expandable implants may assist with restoration of proper loading to the spinal anatomy. Implants which include insertion and expansion members that have a narrow profile, may also provide clinical advantages. In some circumstances, it is desirable to have vertebral endplate contacting surfaces that effectively spread loads across the vertebral endplates. Vertebral body implants may also include a member for maintaining the desired positions, and in some situations, being capable of collapsing. Fusion implants including one or more openings may also be advantageous because they allow for vascularization and bone growth through the implant.

The implant commonly used following a discectomy is an interbody fusion device, also referred to in the art as a cage. Conventional cage designs have a cylindrical or rectangular shape, supporting walls, and a hollow interior space for receiving grafting materials. Cylindrical cages typically have threads along their entire length, whereas rectangular cages have serrated anchors on the upper and lower surfaces. Threaded cylinders usually have small pores and graft material is located inside the hollow interior of the cylinder. The rigid hollow design of fusion cages provide sufficient construct stiffness in arthrodesis and affords a substantial stability for the motion segments after spinal surgery, as well as shielding stress on the implanted graft. Boden S, et al., Spine 20:102 S-112S (1995); Silva M J, et al., Spine, 22(2):140-150 (1997). Commercially available interbody fusion devices comprising threaded cages include, for example, the BAK series of interbody fusion devices available from Zimmer Spine Inc, Minneapolis, Minn.), and the INTERFIX Threaded Fusion Device (by Medtronic Sofamor Danek, Memphis, Tenn.); BAK is a registered trademark of Zimmer Spine Inc.

### SUMMMARY

In one aspect of the present invention a spinal implant is provided. The spinal implant comprises: one or more integrated walls spaced apart to define an interior cavity configured to retain bone graft material, and having at least one aperture providing a pathway between the interior cavity and an exterior environment of the implant; wherein at least one of the one or more walls has a lumen therein, the lumen terminating at a post-operatively accessible refill port and having one or more drug delivery ports disposed in an exterior surface of the at least one wall, and configured to allow drugs to flow from the refill port to the one or more drug delivery ports.

In another aspect of the present invention a bone implant is provided. The bone implant comprises: one or more integrated walls spaced apart to define an interior cavity configured to retain bone graft material, and having at least one aperture providing a pathway between the interior cavity and an exterior environment of the implant; wherein at least one of the one or more walls has a lumen therein, the lumen terminating at a post-operatively accessible refill port and having one or more drug delivery ports disposed in an exterior surface of the at least one wall, and configured to allow drugs to flow from the refill port to the one or more delivery ports.

In another aspect of the present invention a spinal implant system is provided. The spinal implant system comprises: a spinal implant having: one or more integrated walls spaced apart to define an interior cavity configured to retain bone graft material, and having at least one aperture providing a pathway between the interior cavity and an exterior environment of the implant, wherein at least one of the one or more walls has a lumen therein, the lumen terminating at a post-operatively accessible refill port and having one or more drug delivery ports disposed in an exterior surface of the at least one wall, and configured to allow drugs to flow from the refill port to the one or more delivery ports; and a drug source fluidically coupled to the refill port of the spinal implant.

In another aspect of the present invention, a method of using a spinal implant comprising one or more integrated walls spaced apart to define an interior cavity configured to retain bone graft material, and having at least one aperture providing a pathway between the interior cavity and an exterior environment of the implant, wherein at least one of the one or more walls has a lumen therein, the lumen terminating at a post-operatively accessible refill port and having one or more drug delivery ports disposed in an exterior surface of the at least one wall is provided. The method comprises: implanting the spinal implant into a vertebral body of a patient; fluidically coupling the refill port to a drug source; and delivering drugs from the drug source to the refill port to facilitate the flow of drugs from the refill port to the one or more delivery ports.

### BRIEF DESCRIPTION OF THE DRAWINGS

Illustrative embodiments of the present invention are described herein with reference to the accompanying drawings, in which:

FIG. 1 is a perspective view of a segment of a human spinal column;

FIG. 2 is a perspective view of a vertebral body implant in accordance with embodiments of the present invention;

FIG. 3 is a top view of a vertebral body implant illustrated in FIG. 2;

FIG. 4 is a cross-sectional view of the vertebral body implant illustrated in FIG. 2 taken along section line 4-4;

FIG. 5 is a cross-sectional view of the vertebral body implant illustrated in FIG. 2 taken along section line 5-5;

FIG. 6 is a cross-sectional view of the vertebral body implant illustrated in FIG. 2 taken along section line 6-6;

FIG. 7 is a cross-sectional view of the vertebral body implant illustrated in FIG. 2 taken along section line 7-7;

FIG. 8A is a perspective view of an alternative embodiment of the vertebral body implant illustrated in FIG. 2 depicted with an extension that, when joined to the vertebral body implant, increases the length of the implant, in accordance with embodiments of the present invention;

FIG. 8B is a perspective view of the vertebral body implant and extension illustrated in FIG. 8A joined together, in accordance with embodiments of the present invention;

FIG. 8C is a cross-sectional view of the vertebral body implant and extension illustrated in FIG. 8B taken along section line 8C-8C;

FIG. 9 is a side view of a pedicle screw in accordance with embodiments of the present invention;

FIG. 10 is a cross-sectional view of the pedicle screw illustrated in FIG. 9 taken along section line 10-10;

FIG. 11 is a top view of an implanted arrangement of two pedicle screws illustrated in FIG. 9, in accordance with embodiments of the present invention;

FIG. 12 is a perspective view of the vertebral body implant illustrated in FIG. 2 implanted in a vertebral body, in accordance with embodiments of the present invention;

FIG. 13 is a side view of a cartilage implant implanted in the intevertebral disc adjacent the human pelvis, in accordance with embodiments of the present invention;

FIG. 14 is a flowchart illustrating a method for implanting an embodiment of a vertebral body implant in accordance with embodiments of the present invention; and

FIG. 15 is a perspective view of a spinal implant system in accordance with embodiments of the present invention.

### DETAILED DESCRIPTION

Aspects and embodiments of the present invention are directed to a medical implant implantable in the cartilage or bone of a patient to provide long-term replenishable local administration of a drug to the bone or cartilage at the implant site. Embodiments of the present invention are described below with reference to medical implants implantable in the bone and cartilage of the spinal column. Such implants are generally and collectively referred to herein as spinal implants.

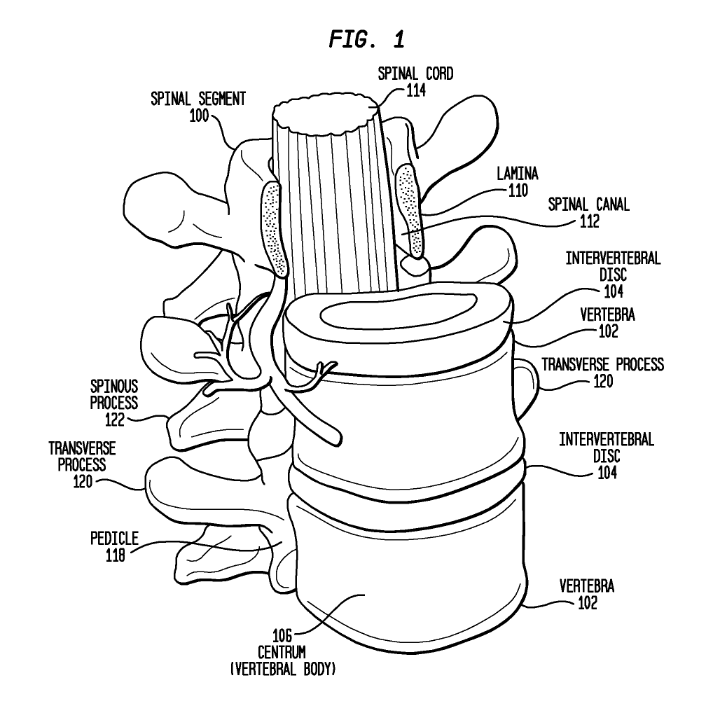

Referring to FIG. 1, vertebral corpectomy is a surgical procedure that involves removing a portion of a vertebral body 106 in cases of, for example, trauma, infection (osteomyelitis), and spinal metastases, etc. A discectomy is a surgical procedure in which the central portion of an intervertebral disc 104, the nucleus pulposus, is removed. A discectomy is often performed in connection with a vertebral corpectomy, although there are a variety of degenerative and other diseases of intervertebral discs 104 which may require a discectomy. A pathological fracture or surgical resection of a vertebral body 106 or intervertebral disc 104 adversely affects the ability of the bone or disc to structurally support the patient's spinal column. As such, corpectomies and discectomies usually require reconstruction of the resected portion of the vertebra 102 or disc 104.

Certain aspects and embodiments of the present invention are generally directed to an improved spinal implant, a vertebral body implant that restores the biomechanical integrity of the spinal column while enabling in vivo delivery of drugs to vertebral body 106. Regarding the restoration of biomechanical integrity, embodiments of the vertebral body implant are constructed to contact the intervertebral disc 104 or vertebra 102 above and below the corpectomized vertebra 102, and to structurally transfer the load placed on the implant. In some embodiments, the vertebral body implant retains bone growth promoting materials which interface with vertebral body 106 to strengthen the bone and/or to integrate the vertebral body implant into the vertebral body.

Regarding the in vivo delivery of drugs to the vertebral body, embodiments of the vertebral body implant of the present invention may be used to deliver a range of different synthetic or naturally occurring pharmaceutical or biological agents (collectively and generally referred to as ‘drugs” herein) in liquid or gel formulations depending upon the particular application. Such drugs may be administered for any actual or potential therapeutic, prophylactic or other medicinal purpose. Representative examples of drugs which may be released from embodiments of a bone or cartilage implant of the present invention include but are not limited to analgesics, anesthetics, antimicrobial agents, antibodies, anticoagulants, antifibrinolytic agents, anti-inflammatory agents, antiparasitic agents, antiviral agents, cytokines, cytotoxins or cell proliferation inhibiting agents, chemotherapeutic agents, radiolabeled compounds or biologics, hormones, interferons, and combinations thereof. Thus, it is contemplated that implants of the present invention may be used to deliver a formulation comprising an agent used in chemotherapy, radiotherapy (brachytherapy or a radioactive substrate including, but not limited, to a liquid or gel).

Alternatively, implants of the present invention may used to deliver drug(s) used in the management of pain and swelling that occurs following the implantation surgery. For example, an implant may release an effective amount of an analgesic agent alone or in combination with an anesthetic agent. As yet another alternative, the implants of the present invention may used to deliver drug(s) which help minimize the risk of infection following implantation. For example, the implant may release a therapeutic or prophylactic effective amount one or more antibiotics (for example, cefazolin, cephalosporin, tobramycin, gentamycin, etc.) and/or another agent effective in preventing or mitigating biofilms (for example, a quorum-sensing blocker or other agent targeting biofilm integrity). Bacteria tend to form biofilms on the surface of implants, and these biofilms, which are essentially a microbial ecosystem with a protective barrier, are relatively impermeable to antibiotics. Accordingly, systemically administered antibiotics may not achieve optimal dosing where it is most needed. However, embodiments of the implant enable the delivery of the desired dose of antibiotic precisely when and where needed. In certain circumstances, the antibiotic may be delivered beneath the biofilm.

Certain embodiments of the bone and cartilage implants of the present invention are adapted for use in the treatment of bone metastases, and in specific embodiments of a spinal implant, spinal metastases. In such embodiments, the spinal implant is configured to deliver pharmacological compounds or other drugs used in the treatment of spinal metastases. As noted, spinal metastases are treated surgically by resection, resulting in the removal of significant amounts of bone and soft tissue. Care must also be taken during resection to avoid spilling the tumor d which may cause seeding of tumor cells into surrounding tissue. Embodiments of the spinal implant are configured to locally release one or more chemotherapeutic agents into the surrounding tissue following implantation into vertebra 102 to destroy tumor cells remaining at the surgical site following resection. Utilization of a spinal implant of the present invention may be as a complement or replacement for the systemic chemotherapy and/or radiation therapy that typically is prescribed for such a patient.

As noted above, embodiments of the spinal implant may be used to deliver one or a combination of therapeutic agents, including chemotherapeutic agents (for example, paclitaxel, vincristine, ifosfamide, dacttinomycin, doxorubicin, cyclophosphamide, and the like), bisphosphonates (for example, alendronate, pamidronate, clodronate, zoledronic acid, and ibandronic acid), analgesics (such as opoids and NSAIDS), anesthetics (for example, ketoamine, bupivacaine and ropivacaine), tramadol, and dexamethasone. In other variations of these embodiments, the implant is useful for delivering an agent useful in radiotherapy (brachytherapy or a radioactive substrate).

Thus, as an alternative to systemic administration of radioactive agents that are capable of targeting a particular tissue, antigen, or receptor type, these radioactive agents are administered locally following implantation of the implant of the present invention. Such radiotherapy agents include radiolabeled antibodies, radiolabeled peptide receptor ligands, or any other radiolabeled compound capable of specifically binding to the specific targeted cancer cells.

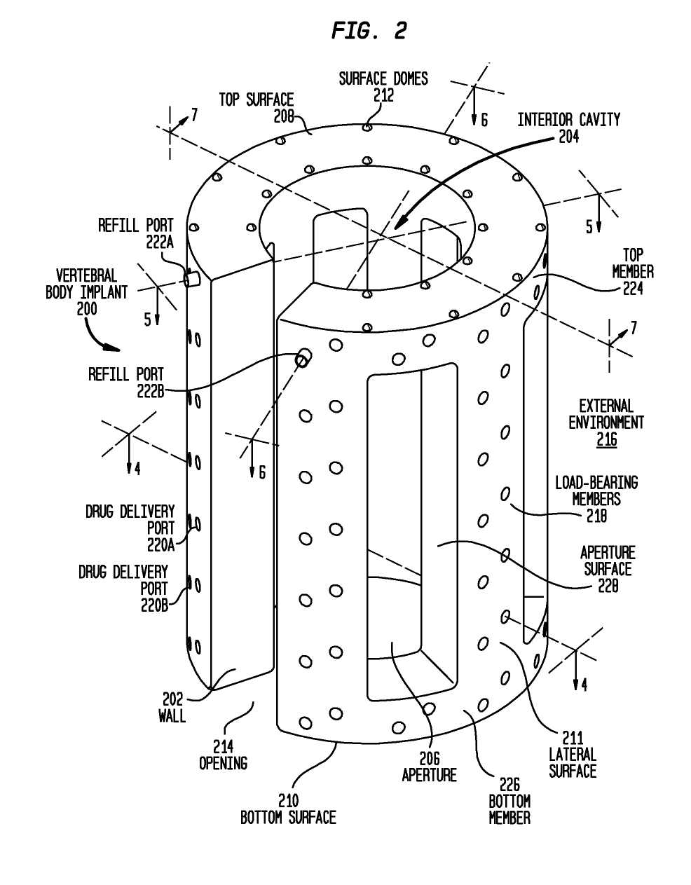

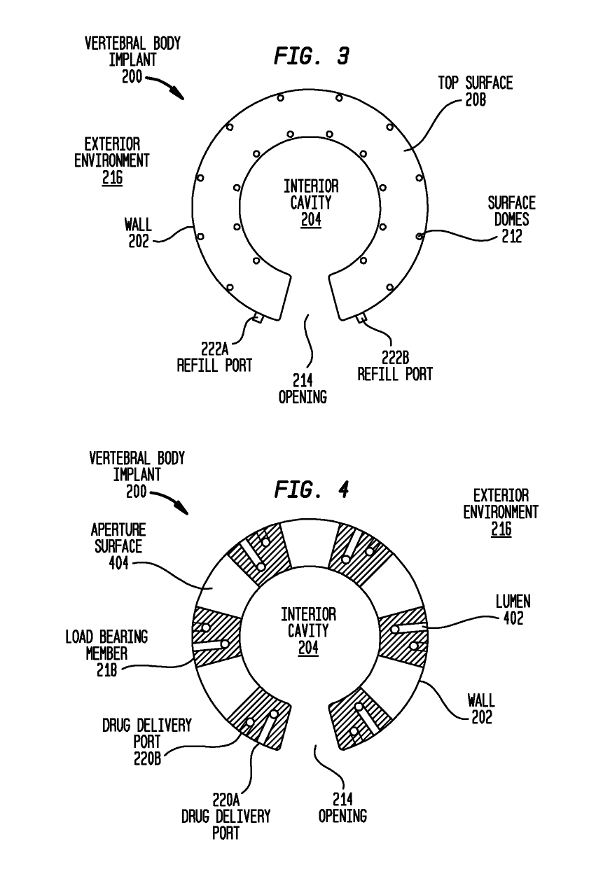

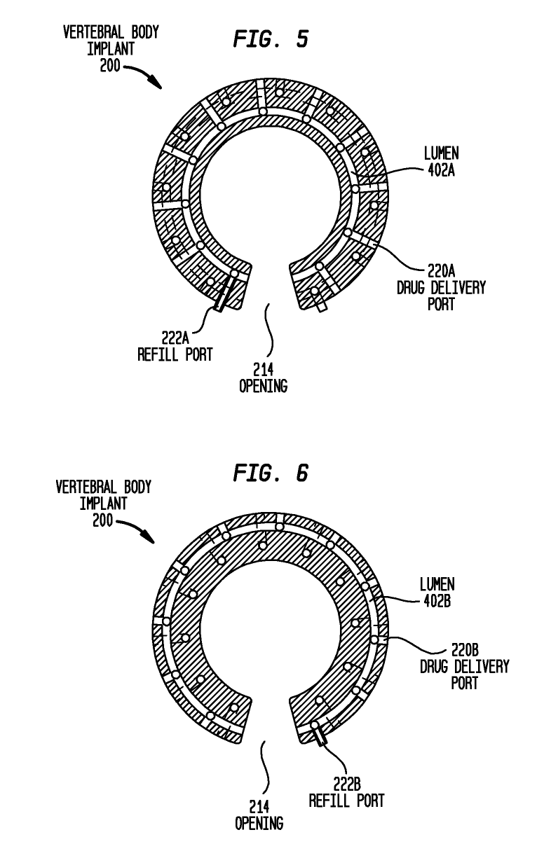

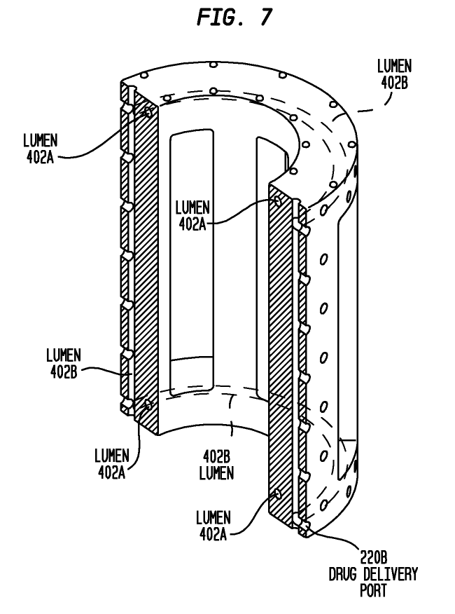

FIGS. 2-7 are different views of embodiments of a bone or cartilage implant of the present invention. Specifically, FIGS. 2-7 illustrate embodiments of a spinal implant for implantation in vertebral body 106 (FIG. 1) of a vertebra 102 (FIG. 1), referred to herein as vertebral body implant 200. In accordance with the teachings of the present invention, vertebral body implant 200 may be used to provide long-term, replenishable delivery of drugs to vertebral body 106 of the vertebra 102 in which it is implanted.

Vertebral body implant 200 comprises a wall 202 configured to encircle or enclose a volume of space referred to herein as interior cavity 204. In the illustrative embodiments of FIGS. 2-7, vertebral body implant 200 is a unitary structure; that is, it is formed of a single cylindrical wall 202. It should be appreciated that in alternative embodiments vertebral body implant 200 may be formed of a plurality of integrated walls 202 spaced from each other to form interior cavity 204.

Interior cavity 204 is configured to retain osteogenic or bone growth promoting materials (collectively and generally referred to herein as bone growth promoting materials; not shown in FIGS. 2-7). Bone growth promoting materials which may be loaded into interior cavity 204 include, but are not limited to, bone morphogenic protein (BMP), bone graft material, bone chips or bone marrow, synthetic or natural autograft, allograft, xenograft, synthetic and natural bone graft substitutes such as bioceramics and polymers, osteoinductive factors, a demineralized bone matrix (DBM), mesenchymal stem cells, a LIM mineralization protein (LMP), or any other suitable bone growth promoting material or substance that would occur to one of skill in the art. It would be appreciated that the bone growth promoting material may be used with or without a suitable carrier to aid in maintaining the material within the device. These carriers can include collagen-based carriers, bioceramic materials, such as BIOGLASS, hydroxyapatite and calcium phosphate compositions; BIOGLASS is a registered trademark of the University of Florida, Gainesville Fla. The carrier material may be provided in the form of a sponge, a block, folded sheet, putty, paste, graft material or other suitable forms. The bone growth promoting material may be provided in a composition that includes an effective amount of a bone morphogenetic protein (BMP), transforming growth factor βI, insulin-like growth factor I, platelet-derived growth factor, fibroblast growth factor, LIM mineralization protein (LMP), and combinations thereof or other therapeutic or infection resistant agents. Additionally, the bone growth promoting material may be resorbable or nonresorbable. Examples of resorbable materials that may be used include, but are not limited to, polylactide, polyglycolide, tyrosine-derived polycarbonate, polyanhydride, polyorthoester, polyphosphazene, calcium phosphate, hydroxyapatite, bioactive glass, and various combinations thereof.

A manual access opening 214 in vertebral body implant 200 provides the ability of a surgeon or other medical professional to manually access interior cavity 204 to, for example, place bone growth promoting material into the cavity. Access opening 214 may have any form suitable for the dimensions of the implant, the viscosity of the bone growth promoting material, and other factors. In the embodiment illustrated in FIG. 2, unitary wall 202 is does not form a concentric circle, but rather contains a discontinuity therein defining opening 214. Additionally, wall 204 has a top surface 208 and a bottom surface 210 configured to abut cartilage and/or bone when implanted in a patient. In certain embodiments top and/or bottom surfaces 208 and 210 have a surface finish or surface features which facilitate placement and/or prevent lateral movement or dislodgement of the implant during implantation. In the embodiments illustrated in FIGS. 2-7, these surface features comprise surface domes 212 on top surface 208.

A plurality of apertures 206 are disposed in wall 202. Apertures 206 each provide an open pathway through which interior cavity 204 communicates with an exterior environment 216 of implant 200. In the embodiments illustrated in FIGS. 2-7, five (5) apertures 206 are circumferentially spaced around cylindrical wall 202. It should be appreciated, however, that more or fewer apertures may be provided in other embodiments. Furthermore, such aperture(s) 206 may have any dimensions suitable for enabling the bone growth promoting material retained in interior cavity 204 to interact with bone, which is proximate to implant 200, and which do not compromise the intended function of the implant. For example, in exemplary vertebral body implant 200 of FIGS. 2-7, wall(s) 204 are constructed and arranged such that the portions of wall 202 extending between top and bottom surfaces 208, 210, and which are laterally adjacent to apertures 206, referred to below as load bearing members 218, are capable of bearing the load placed on top surface 208 of implant 200, thereby retaining the structural integrity necessary to support the spinal column.

Vertebral body implant 200 further comprises a drug delivery network for the replenishable in vivo delivery of drugs to the vertebral body. The drug delivery network comprises a refill port 222A, a lumen 402A (FIGS. 4-7) in wall 202, and drug delivery ports 220A. Lumen 402A is a continuous passageway in wall 202 which terminates at, and fluidically couples, refill port 222A and drug delivery ports 220A.

In the embodiments illustrated in FIGS. 2-7, there are two such drug delivery networks implemented in vertebral body implant 200. The first drug delivery network comprises, as noted, refill port 222A, lumen 402A and drug delivery ports 220A. The second drug delivery network comprises refill port 222B, lumen 402B and drug delivery ports 220B.

In these embodiments, two drug delivery networks are independent of each other. That is, as shown in FIGS. 4-7, the two drug delivery networks are not fluidically coupled to each other. As such, drugs introduced into refill port 222A will travel only through lumen 402A and be delivered only via ports 220A. Similarly, a drug introduced into refill port 222B will travel only through lumen 402B and will be delivered only via ports 220B. Implementing multiple drug delivery networks in a single bone or cartilage implant provides for the independent administration of multiple drugs. In addition, each such independent drug delivery network may be coupled to a different source of such drugs.

In the embodiments illustrated in FIGS. 2-7, lumens 402A and 402B are each a contiguous lumen traveling through load bearing members 218 as well as the top member 224 and bottom member 226 of implant 200. It should be appreciated that in alternative embodiments there may be a single drug delivery network, implemented in the bone or cartilage implant. It should also be appreciated that the network may be located in any portion of the implant suitable for delivering a therapeutic dosage of a selected drug to a target location.

Drug delivery ports 220 may be disposed on top surface 208, bottom surface 210, lateral surface 211, and aperture surfaces 228. It should be appreciated, however, that any such surfaces may have no drug delivery ports 220. The distribution of ports 220 may be achieved at the time of fabrication or following fabrication by occluding specific ports with, for example, a plug or epoxy.

Additionally, the size of drug delivery ports may vary. In certain embodiments, drug delivery ports 220 may have a diameter of approximately 250-500 microns. Drug delivery ports 220 of this size may be expected to provide optimal bone ingrowth. In one embodiment, to provide further bone ingrowth, a portion, e.g., a portion of the tissue- or bone-mating surfaces, of the prosthesis is porous. Thus, the porous portion is a tissue-contact surface that facilitates ingrowth and provides stable fixation of the implant in the body. In another embodiment, the entire surface of implant 200 is porous.

As noted, implant 200 comprises refill ports 222 through which drugs may be introduced and reintroduced into implant 200. In certain embodiments, refill ports 222 are configured to be detachably connected to a catheter (not shown), the opposing end of which is fluidically connected to a drug source such as a syringe port, an active drug or programmable infusion device, or a passive drug infusion device. As noted, refill ports 222 communicate with a respective lumen which, in turn, communicates with a plurality of drug delivery ports 220 located at selected locations on the surface of implant 200. The lumens may optionally contain a porous inner substrate (not shown) such as silica or polymer beads tailored to facilitate diffusion of a drug.

In certain embodiments, wall 202 of implant 200 may be formed of, be coated with, or otherwise comprise a biocompatible material selected from metals, polymers, ceramics, and combinations thereof. Typically, embodiments of the present invention are non-biodegradable since the implant is intended to function in a patient for an extended period, preferably for the life of the patient. For instance, in certain embodiments, wall 202 of implant 200 may be formed from a stainless steel, a chrome-cobalt alloy, a titanium alloy, a ceramic, an ultra high molecular weight polyethylene (e.g., a highly cross-linked, UHMW polyethylene), or PEEK and PEEK composites. In other embodiments, the implant is formed of or includes a ceramic (e.g., alumina, silicon nitride, zirconium oxide), a semiconductor (e.g., silicon), a glass (e.g., Pyrex, BPSG), or a degradable or non-degradable polymer; Pyrek is a trademark of Corning Inc, New York.

In the embodiments of FIGS. 2-7, vertebral body implant 200 is, as noted above, cylindrical in shape. However, it would be appreciated by those or ordinary skill in the art that implant 200, or other bone and cartilage implants of the present invention, may have other shapes and sizes which also provide the requisite mechanical support. Exemplary shapes include, for example, a rectangle, sphere, dome, or other shape.

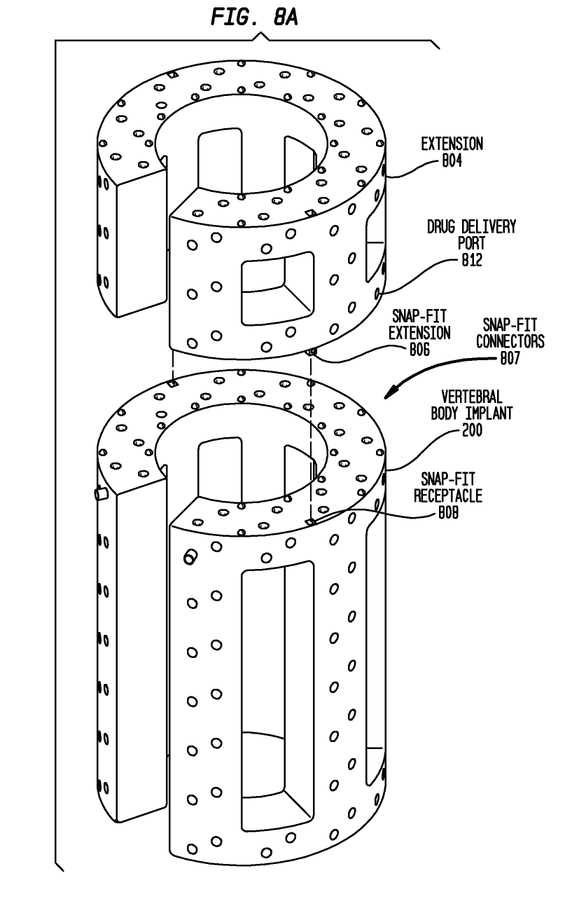

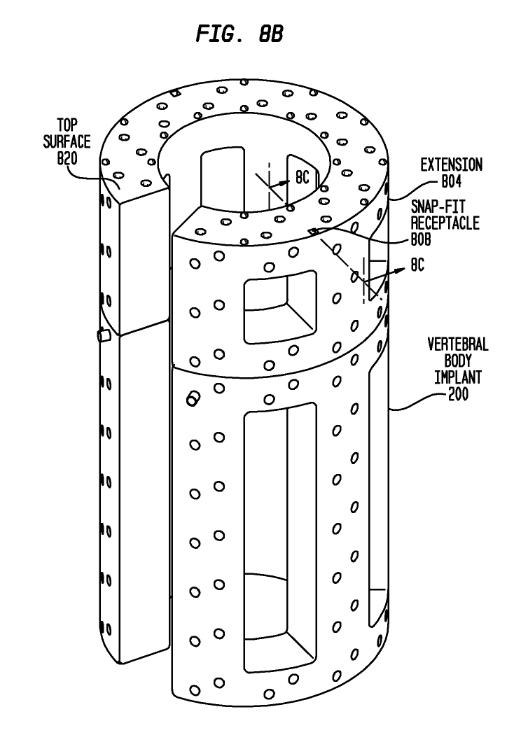

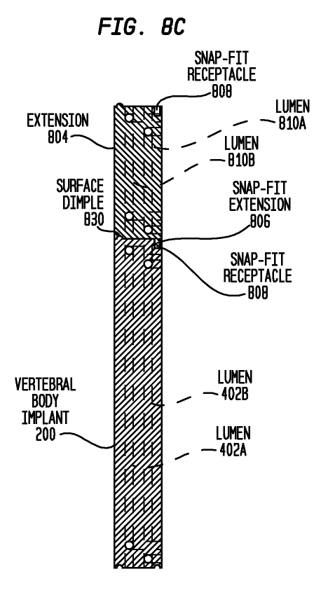

Vertebral body implant 200 may be one of a plurality of vertebral body implants each dimensions to accommodate a particular corpectomy or vertebra size. In such embodiments the surgeon will have the opportunity select the vertebral body implant having the size most appropriate for the particular corpectomy. However, in certain circumstances, surgical resection may result in the removal of relatively large regions of a vertebral body 106 such an implant may not properly fit with the resected region. For example, a selected implant 200 may be too small to provide the desired structural support and the top surface 208 and bottom surface 210 of vertebral body implant 200 are unable to simultaneously contact vertebra 102 or disc 104 immediately above and below the resected region. FIGS. 8A-8C are perspective and cross-sectional views of other embodiments of the present invention configured to resolve such sizing issues.

In the embodiments illustrated in FIGS. 8A-8C, an extension 804 is provided for attachment to top surface 208. Extension 804 has a cross-sectional profile which is the same as the cross-sectional profile of implant 200. In addition, an interlocking mechanism may be provided to facilitate the secure joining of extension 804 to vertebral body implant 200. In the illustrative embodiments shown in FIGS. 8A-8C, the interlocking mechanism is implemented as one or more snap-fit connectors 807 each comprising one or more snap-fit extensions 806 extending from extension 804, and one or more corresponding snap-fit receptacles 808 within top surface 210 of implant 200. Snap-fit receptacles 808 are each configured to receive and mate with a snap-fit extension 806 causing extension 804 to be securely joined to implant 200. This is best illustrated in FIG. 8B. In the illustrative embodiments, two snap-fit connectors 807 are located on opposing sides of the surfaces of extension 804 and implant 200.

It should be appreciated that additional extensions may be added to the implant illustrated in FIG. 8B. This is illustrated by the snap-fit receptacles 808 disposed in top surface 820 of implant extension 804.

In the embodiments shown in FIGS. 8A-8C, extension 804 has lumens 810 and drug delivery ports 812 which operate substantially similar to the lumens and ports described above with reference to FIGS. 2-7. As shown in FIG. 8C, in certain embodiments, drug-delivery ports 220A and 220B on top surface 208 are configured to aligned with ports on the bottom surface of extension 804. As such, lumens 402A and 402B are aligned with, and fluidically coupled to, lumens 810A, 810B in extension 804 so that drugs may be delivered through ports 812.

As noted above, tope surface 208 may have surfaces domes 212 disposed thereon. As shown in FIG. 8C, surface domes 212 on top surface 208 of implant 200 are aligned and mate with corresponding surface dimples 830 in the bottom surface of extension 804 to insure there is a flush mating surface between implant 200 and extension 804.

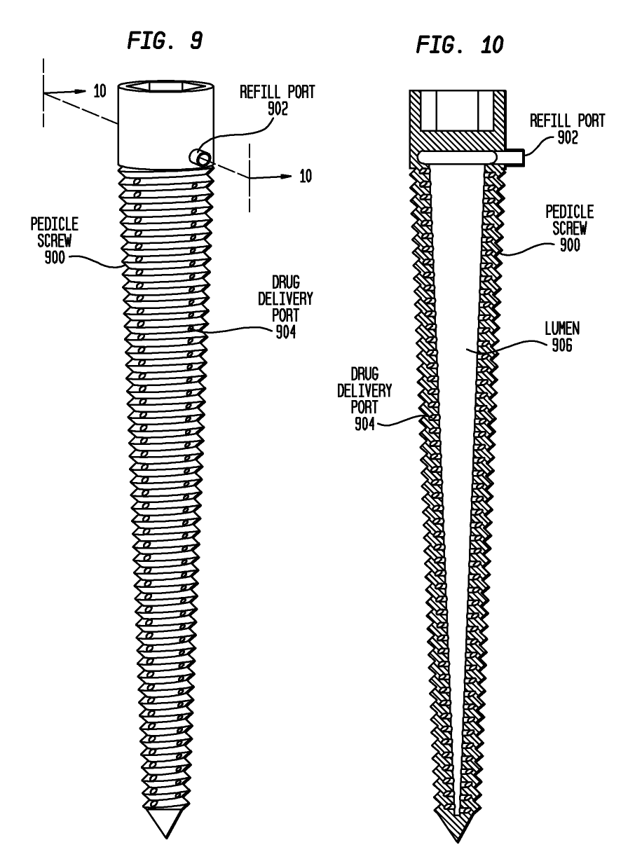

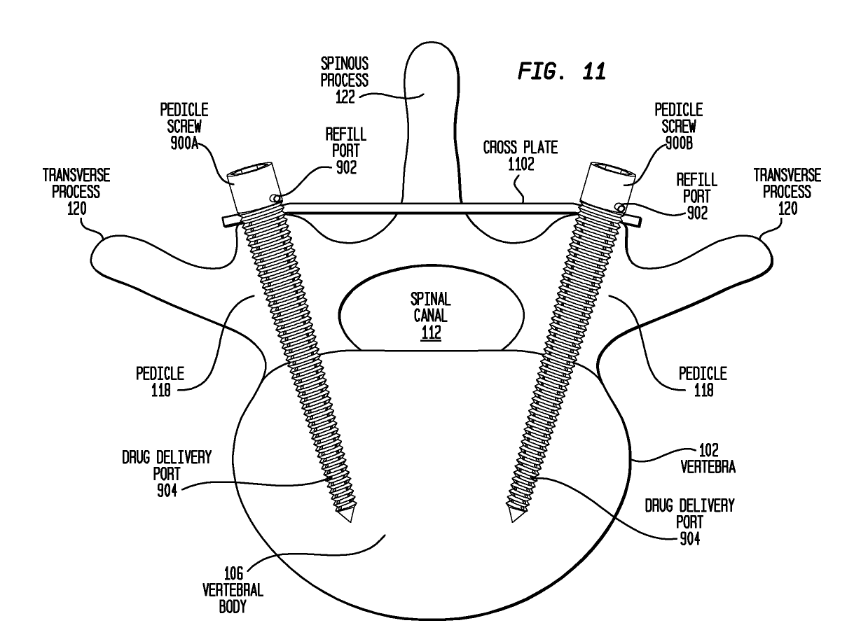

FIG. 9 is a side view of an alternative embodiment of the present invention implemented as a pedicle screw 900. FIG. 10 is cross-sectional view of pedicle screw 900 of FIG. 9, taken along cross-sectional line 10-10. In the illustrative embodiments, each pedicle screw comprises a refill port 902, lumen 906 and drug delivery ports 904. These elements of pedicle screw 900 function substantially similar to the analogous elements described above with reference to vertebral body implant 200.

As is well-known in the art, pedicle screws are typically implemented as a part of a larger implantable structural support system. FIG. 11 illustrates two pedicle screws 900 of FIGS. 9-10 each inserted into pedicle 118 of a vertebra 102. As shown, screws 900 are connected to one another by a cross plate 1102 forming part the larger structural support system.

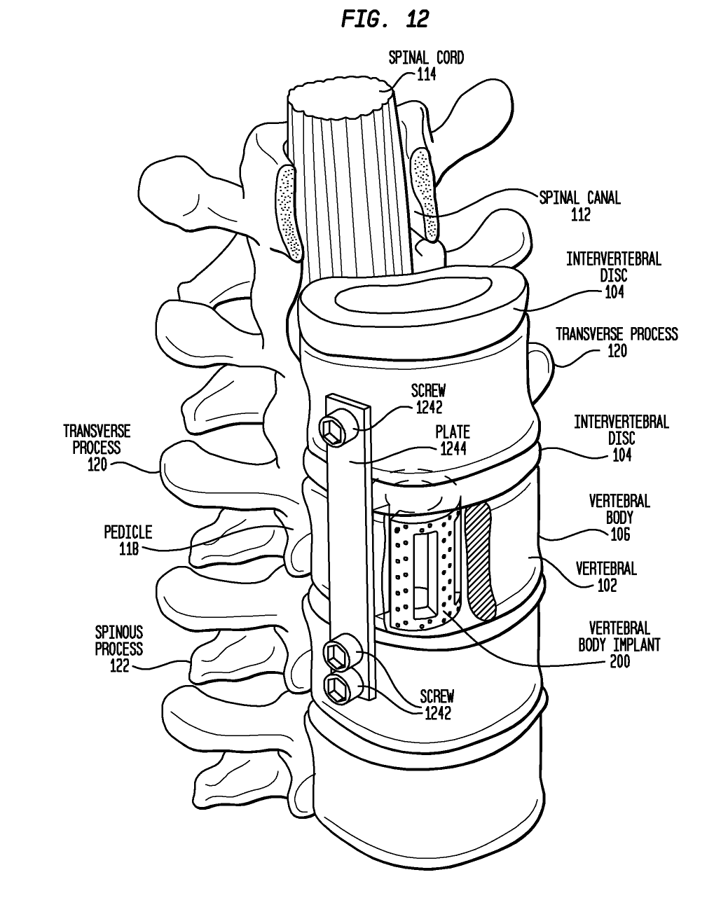

As noted above, vertebral body implant 200 may be configured for bone ingrowth, or may have surface features which prevent movement of the implant. In certain circumstances, additional stabilization of vertebral body implant 200 is desired. FIG. 12 illustrates an embodiment in the additional stabilization is provided by a biocompatible, photo-initiated polymer rod or plate 1244. In this illustrative arrangement, plate 1244 is secured to vertebral body implant 200, as well as healthy vertebrae above and below the damaged site. As shown, plate 1244 is secured to the healthy vertebrae by screws 1242. Additionally, guide plates may be provided for drilling holes to affix plate 1244 and/or rods to the vertebrae with the necessary screws. Such screws may be bone screws or pedicle screws, such as pedicle screws 900 of FIGS. 9-11. In specific cases, the additional stabilization may employ currently available rigid devices for such purposes with screws that are compliant or non-compliant. An example of a suitable screw and plate fixation device which may used with the present invention is the Kaneda Device (by DePuy-Acromed, Cleveland Ohio).

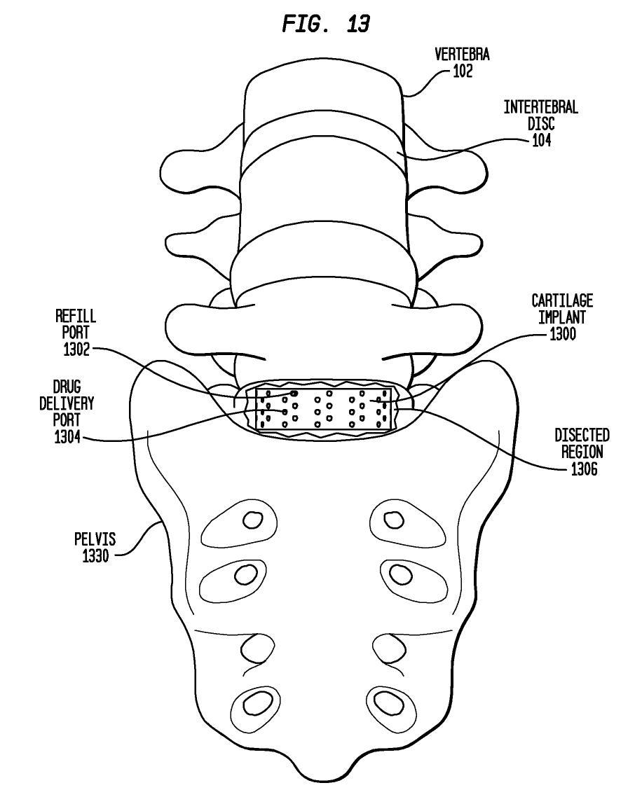

FIG. 13 illustrates another bone and cartilage implant of the present invention, shown as cartilage implant 1300. Cartilage implant 1300 is configured similar to implant 200 described above, but is dimensioned to be implanted in a resected region of an intervertebral disc. In the specific embodiment of FIG. 13, cartilage implant 1300 is dimensioned to be positioned in resected region 1306 of an intervertebral disc adjacent pelvis 1330. In the perspective illustrated in FIG. 13, a refill port 1302 and drug deliver ports 1304 are visible.

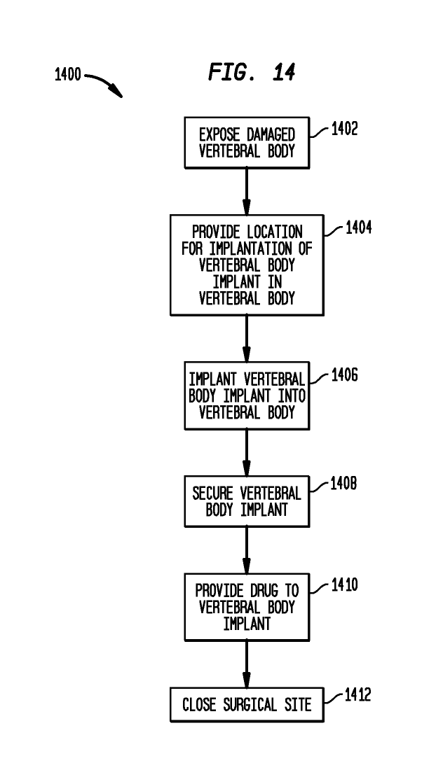

FIG. 14 is a flowchart illustrating a method 1400 for using a spinal implant, and particularly a vertebral body implant, in accordance with embodiments of the present invention. At block 1402, a surgeon exposes a damaged vertebral body of the patient. Exposing the vertebral body includes administering general anesthesia to the patient, and properly positioning the patient for access to the damaged vertebral body. A standard anterior thoracic or lumbar approach, or a lateral extracavitary approach may then be used to expose the vertebral body.

At block 1402, the surgeon provides a location for implantation of the vertebral body implant. This may include performing a corpectomy by use of a drill or bone ronguers to remove damaged bone. In such embodiments, this step further includes ensuring that the proper amount of bone has been removed by checking the depth of the newly created corpectomy cavity using a marker and intraoperative X-ray. Once the desired depth is achieved, osteotomes and a drill with cutting burr are used to enlarge the corpectomy cavity. Under fluoroscopic guidance, distraction is applied to the vertebral bodies above and below the corpectomy cavity, and a ruler is used to measure the corpectomy cavity to ensure that the cavity is a proper size to receive the vertebral body implant.

At block 1406, the vertebral body implant is implanted into the corpectomy cavity. Prior to implantation, morsellized bone allograft or calcium triphosphate, prepared as per protocol, is placed into the interior of the vertebral body implant. The vertebral body implant is then impacted into the corpectomy cavity using tamps and a mallet, and then countersunk to sit into the midportion of the cavity. Fluoroscopic or other imaging may be used to ensure that the vertebral body implant is properly positioned. Once this is completed, distraction of the vertebral bodies above and below the corpectomy cavity is released.

At block 1408, the vertebral body implant is secured to the patient. The vertebral body implant may be secured using the surface features provided thereon, or through the use of, for example, a fixation system as illustrated in FIG. 12. As noted, if a fixation system is used, the implant is connected to a plate which is attached to healthy vertebra using screws. Once the screws are inserted, AP and lateral X-rays are obtained to confirm proper placement of the vertebral body implant, screws and any other hardware.

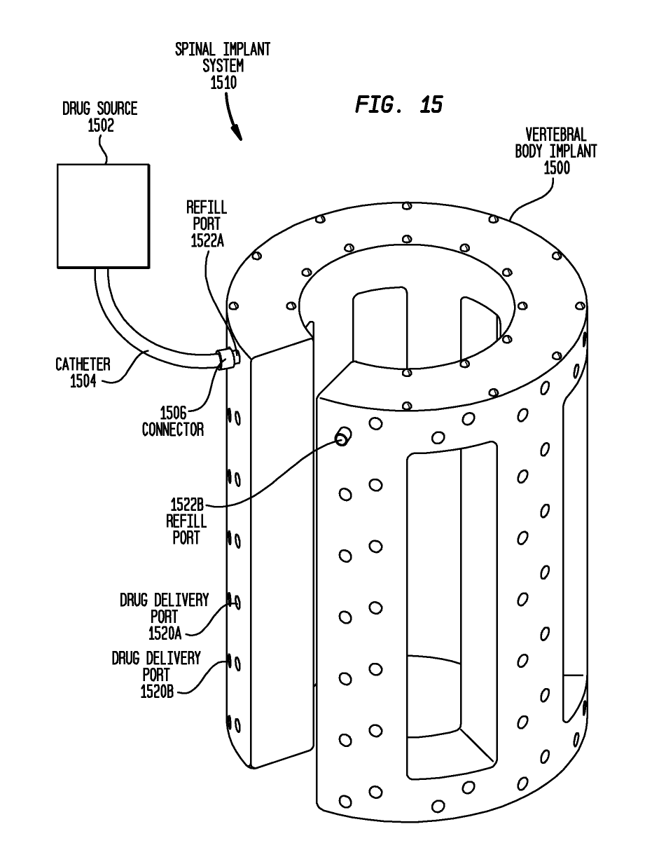

In the embodiment of FIG. 14, at block 1410 a drug is provided to the implant. This may include, for example, using a syringe to fill the vertebral body implant, or connecting the vertebral body implant to drug source such as described below with reference to FIG. 15.

At block 1412, the surgical site is closed. This may include irrigating the area and closing the incision per standard surgical techniques.

It would also be appreciated that the illustrative surgical method of FIG. 14 is merely exemplary, and various modifications to the method are within the scope of the present invention. For example, in embodiments of the present invention, the drug may be provided to the vertebral body implant before or after implantation. Additionally, there are a number of methods by which drugs may be introduced to the implant, and by which the drug flows may from the implant through the drug delivery ports. For example, as noted above, drugs may be introduced under pressure using a syringe to facilitate flow of drugs from the delivery ports. In other embodiments described below, a reservoir pump may facilitate the flow of drugs from the delivery ports. Additionally, capillary action may be used to facilitate the flow of drugs from the delivery ports. It would be appreciated that these examples are provided for illustration and do not limit the embodiments of the present invention.

Furthermore, in certain circumstances, the drug is not necessarily provided prior to closure of the surgical site. Specifically, as discussed above, the vertebral body implant includes a port that is post-operatively accessible. As such, this port could be used to provide the drug to the vertebral body implant after surgical site closure.

As noted above, bone and cartilage implants of the present invention are configured to deliver drugs to a patient. FIG. 15 illustrates a spinal implant system 1510 of the present invention that includes a vertebral body implant 1500 connected to an implantable drug source 1502. Similar to the embodiments described above, vertebral body implant 1500 comprises refill ports 1522A and 1522B, as well as a plurality of respective drug delivery ports 1520A, 1520B. As shown, a connector 1506 detachable connects refill port 1522A to the distal end of a catheter 1504 extending from drug source 1502. Catheter 1504 may comprise any catheter now known or later developed, and connector 1506 may comprise any device which detachably couples the catheter to refill port 1522A. In embodiments of the present invention, drug source 1502 may include a reservoir (not shown) and a post-operatively accessible refill port (also not shown).

In certain embodiments of the present invention, drug source 1502 is an active drug infusion device, such as the Medtronic SYNCHROMED programmable pump; SYNCHROMED is registered trademark of Medtronic Inc., Minneapolis Minn. Such pumps typically include a drug reservoir, a peristaltic pump to pump the drug from the reservoir, and a catheter port to connect the source to a catheter. Such devices also typically include a battery to power the pump, an electronic module to control the flow rate of the pump, and possibly an antenna to permit the remote programming or control of the pump. It should be appreciated that the pump may be implanted in, or secured externally to, the patient.

In alternative embodiments of the present invention, drug source 1502 comprises a passive drug infusion device that does not include a pump. In one such embodiment, drug source 1502 includes a pressurized reservoir that delivers the drug to refill port 1540 via catheter 1504. Such passive drug infusion devices are generally smaller and less costly than active drug infusion devices. An example of a passive device that may be used with embodiments of the present invention is the Medtronic ISOMED; ISOMED is registered trademark of Medtronic Inc., Minneapolis Minn. This device delivers a drug via a reservoir which is pressurized with a drug to between 20-40 psi. This pressurization is provided by a syringe capable of delivering drugs between 35-55 psi.

In embodiments of the present invention, spinal implant system 1510 is configured to release drugs in various temporal and spatial patterns and profiles, for example, releasing a drug in a continuous or pulsatile manner for several (e.g., 5 to 15) days and/or targeting areas of the implant, if any, that are more conducive to bacterial growth. In further embodiments, drug delivery ports 1520 are controllable to alter the flow rate through the ports. Such control may be provided externally, such as by electrical or mechanical signals, heat, etc.

While embodiments of the invention have been described with a certain degree of particularity, it is understood that the invention is not limited to the embodiments set forth herein for purposes of exemplification. Modifications and variations of the specific methods and devices described herein will be obvious to those skilled in the art from the foregoing detailed description. Such modifications and variations do not depart from the inventive concept and scope of the present invention and are intended to come within the scope of the appended claims.

What is claimed is:

1. A spinal implant comprising:
one or more integrated walls spaced apart to define an interior cavity configured to retain bone graft material, and having at least one aperture providing a pathway between the interior cavity and an exterior environment of the implant;
wherein at least one of the one or more walls has a lumen therein, the lumen terminating at a post-operatively accessible refill port and at one or more drug delivery ports disposed in an exterior surface of the at least one wall, and configured to allow drugs to flow from the refill port to the one or more delivery ports.

2. The spinal implant of claim 1, wherein the one or more integrated walls collectively define top and bottom surfaces and load bearing members extending between the top and bottom surfaces.

3. The spinal implant of claim 2, wherein the top and bottom surfaces are each configured to contact one or more of bone and cartilage.

4. The spinal implant of claim 1, wherein the one or more integrated walls define a substantially cylindrical vertebral body cage implant.

5. The spinal implant of claim 1, wherein the least one aperture comprises a plurality of apertures formed in a spaced arrangement in the one or more integrated walls.

6. The spinal implant of claim 5, wherein the one or more integrated walls define a substantially cylindrical vertebral body cage implant, and further wherein the at least one aperture comprises a plurality of apertures circumferentially spaced around the cylindrical vertebral body cage implant.

7. The spinal implant of claim 1, wherein the at least one wall has a second lumen therein, the second lumen terminating at a second post-operatively accessible refill port and has at one or more second drug delivery ports disposed in an exterior surface of the at least one wall, and configured to allow drugs to flow from the refill port to the one or more second delivery ports.

8. The spinal implant of claim 1, wherein the refill port is configured to securely and detachably connect to a catheter.

9. The spinal implant of claim 1, wherein the spinal implant is configured to be implanted in at least one of a vertebral disc or a location of an explanted disc.

10. The spinal implant of claim 1, wherein the spinal implant is a spinal bone implant.

11. The spinal implant of claim 10, wherein the bone implant is a pedicle screw.

12. The spinal implant of claim 10, wherein the bone implant is configured to be implanted in the vertebral body.

13. The spinal implant of claim 1, wherein the flow rate through the one or more drug delivery ports is externally controllable.

14. The spinal implant of claim 2, further comprising:
an extension configured to be detachably connected to the top surface of the one or more integrated walls.

15. A bone implant comprising:
one or more integrated walls spaced apart to define an interior cavity configured to retain bone graft material, and having at least one aperture providing a pathway between the interior cavity and an exterior environment of the implant;
wherein at least one of the one or more walls has a lumen therein, the lumen terminating at a post-operatively accessible refill port and at one or more drug delivery ports disposed in an exterior surface of the at least one wall, and configured to allow drugs to flow from the refill port to the one or more delivery ports.

16. The bone implant of claim 15, wherein the one or more integrated walls collectively define top and bottom surfaces and load bearing members extending between the top and bottom surfaces.

17. The bone implant of claim 16, wherein the top and bottom surfaces are each configured to contact one or more of bone and cartilage.

18. The bone implant of claim 15, wherein the one or more integrated walls define a substantially cylindrical vertebral body cage implant.

19. The bone implant of claim 15, wherein the least one aperture comprises a plurality of apertures formed in a spaced arrangement in the one or more integrated walls.

20. The bone implant of claim 19, wherein the one or more integrated walls define a substantially cylindrical vertebral body cage implant, and further wherein the at least one aperture comprises a plurality of apertures circumferentially spaced around the cylindrical vertebral body cage implant.

21. The bone implant of claim 15, wherein the at least one wall has a second lumen therein, the second lumen terminating at a second post-operatively accessible refill port and has at one or more second drug delivery ports disposed in an exterior surface of the at least one wall, and configured to allow drugs to flow from the refill port to the one or more second delivery ports.

22. The bone implant of claim 15, wherein the refill port is configured to securely and detachably connect to a catheter.

23. The bone implant of claim 15, wherein the bone implant is a spinal bone implant.

24. The bone implant of claim 15, wherein the bone implant is a pedicle screw.

25. The bone implant of claim 15, wherein the bone implant is configured to be implanted in the vertebral body.

26. The bone implant of claim 15, wherein the flow rate through the one or more drug delivery ports is externally controllable.

27. The bone implant of claim 16, further comprising:
an extension configured to be detachably connected to the top surface of the one or more integrated walls.

28. A spinal implant system comprising:
a spinal implant having:
one or more integrated walls spaced apart to define an interior cavity configured to retain bone graft material, and having at least one aperture providing a pathway between the interior cavity and an exterior environment of the implant,
wherein at least one of the one or more walls has a lumen therein, the lumen terminating at a post-operatively accessible refill port and at one or more drug delivery ports disposed in an exterior surface of the at least one wall, and configured to allow drugs to flow from the refill port to the one or more delivery ports; and
a drug source fluidically coupled to the refill port of the spinal implant.

29. The implant system of claim 28, wherein the drug source comprises:
an injection port configured to receive a needle therein.

30. The implant system of claim 28, wherein the drug source comprises:
an implantable fluid reservoir.

31. The implant system of claim 28, wherein the drug source comprises:
an implantable active drug infusion device.

32. The implant system of claim 28, wherein the drug source comprises:
an implantable passive drug infusion device.

33. The implant system of claim 28, wherein the one or more integrated walls collectively define top and bottom surfaces and load bearing members extending between the top and bottom surfaces.

34. The implant system of claim 28, wherein the one or more integrated walls define a substantially cylindrical vertebral body cage implant.

35. The implant system of claim 28, wherein the at least one wall has a second lumen therein, the second lumen terminating at a second post-operatively accessible refill port and has at one or more second drug delivery ports disposed in an exterior surface of the at least one wall, and configured to allow drugs to flow from the refill port to the one or more second delivery ports.

36. The implant system of claim 28, wherein the refill port is configured to be fluidically coupled to the drug source via a catheter.

37. The implant system of claim 36, wherein the catheter is detachable connected to the refill port.

38. The implant system of claim 28, wherein the drug source is externally replenishable.

39. The implant system of claim 28, wherein the spinal implant is a spinal bone implant.

40. The implant system of claim 39, wherein the bone implant is a pedicle screw.

41. The implant system of claim 28, wherein the flow rate through the one or more drug delivery ports is externally controllable.

42. The implant system of claim 29, further comprising:
an extension configured to be detachably connected to the top surface of the one or more integrated walls.

43. A method of using a spinal implant comprising one or more integrated walls spaced apart to define an interior cavity configured to retain bone graft material, and having at least one aperture providing a pathway between the interior cavity and an exterior environment of the implant, wherein at least one of the one or more walls has a lumen therein, the lumen terminating at a post-operatively accessible refill port and at one or more drug delivery ports disposed in an exterior surface of the at least one wall, the method comprising:
implanting the spinal implant into a vertebral body of a patient;
fluidcally coupling the refill port to a drug source; and
delivering drugs from the drug source to the refill port to facilitate the flow of drugs from the refill port to the one or more delivery ports.

44. The method of claim 43, wherein the drug source comprises an externally accessible injection port, and wherein delivering drugs from the drug source comprises:
injecting drugs into the injection port via syringe.

45. The method of claim 43, wherein the drug source comprises an implantable active drug infusion device, and wherein delivering drugs from the drug source comprises:
delivering drugs from the active drug infusion device.

46. The method of claim 43, wherein the drug source comprises an implantable passive drug infusion device, and wherein delivering drugs from the drug source comprises:
delivering drugs from the passive drug infusion device.

47. The method of claim 43, further comprising:
performing a corpectomy to remove damaged portions of the vertebral body; and
inserting the spinal implant into the cavity created by the corpectomy.

48. The method of claim 43, further comprising:
securing the spinal implant to the patient via set screws.
